(19) United States
(12) Patent Application Publication
(10) Pub. No.: US 2008/0208235 A1
(43) Pub. Date: Aug. 28, 2008

(54) Tattoo Machine Tip

(75) Inventors: Christoph Ulmer, Basel (CH); Theodor Burckhardt, Arlesheim (CH)

Correspondence Address: CLEMENTS BERNARD MILLER, 1901 ROXBOROUGH ROAD, SUITE 300, CHARLOTTE, NC, 28211, US

(73) Assignee: Christoph Burckhardt AG, Basel (CH)

(21) Appl. No.: 11/887,681

(22) PCT Filed: Mar. 29, 2006
(86) PCT No.: PCT/EP2006/061126

§ 371 Date: Oct. 2, 2007

(30) Foreign Application Priority Data
Apr. 4, 2005 (EP) 05102643.3

(51) Int. Cl. A61M 37/00; A61B 17/34

(52) U.S. Cl. 606/186

(57) ABSTRACT

A device (10) for removing a tattoo comprises a tattoo machine tip (14) and a tattoo machine (12). The tattoo machine tip (12) comprises a tip body (28) that comprises a needle channel (32) for guiding at least one tattooing needle (34). Said tip body (28) is connected with a supply container (50) for receiving a liquid. According to the invention, said supply container (50) is filled with a skin irritant (52). Further, said supply container (50) is connected through a feed channel for wetting the tattooing needle (34) with said skin irritant (52) with said needle channel (32), wherein said feed channel (54) opens out into said needle channel (32). Unnecessary dead volumes are avoided thereby, so that the consumption of skin irritant is minimized.

The invention relates to a tattoo machine tip for tattoo machines used for removing tattoos.

From EP 1 330 199 B1, a device for removing a tattoo from the human or animal skin has been known. This device has a tattoo machine with at least one needle. For removing the tattoo, said at least one needle is first dipped into a container in which a skin irritant is provided. Subsequently, the needle wetted with said skin irritant pierces the skin in the tattoo area whereby the relatively big color pigment agglomerates of the tattoo are punctured. When the skin is pierced, part of the skin irritant remains in the puncture channel of the needle, so that the comminuted color pigment agglomerates can be ablated outwardly by the natural healing process of the skin.

A drawback of such a tattoo machine is the fact that the dipping of the tattoo machine tip for wetting the needle into the container comprising the skin irritant is tedious and time-consuming. Further, it is required that the needle be wetted throughout the area punctured into the skin in order to avoid too quick a healing of the puncture channel, which would prevent the export of the comminuted color pigment agglomerates. However, when the needle is dipped into the skin irritant, there is a risk that the wetting of the needle is insufficient. This risk is the higher, the lower the filling level in the container is. In order to reduce the risk of insufficient wetting of the needle, it is required that the container is no longer used when it is only partly filled with the skin irritant. Since the removal of tattoos involves high hygienic demands, for example, to avoid infections, the container with the remaining skin irritant cannot be reutilized. Thus, the consumption of skin irritant for removing a tattoo is very high.

From JP 2003 339 875 A, a tattoo machine with a tattoo machine tip has been known. The tattoo machine tip has a tip body essentially having a tubular design with a truncated conical segment at the end of the tattoo machine tip facing towards the skin. Before the tubular portion merges into said truncated conical portion, an opening is provided in said tubular portion for filling ink from a supply container into the tip body. The filled-in ink collects in the truncated conical portion of the tip body where it can wet the needle. Instead of ink, the bleaching agent H₂O₂ may also be filled into the tip body in order to remove tattoos. In order to completely bleach a tattoo, it is required to allow a relatively large amount of liquid to contact with the color pigment agglomerates of the tattoo, so that the amount of liquid introduced into the skin at each prick must be correspondingly high. Thus, the consumption of bleaching agent is relatively high.

From EP 1 495 782 A1, a tattoo machine tip for a tattoo machine has been known that has a lateral supply container filled with ink for tattooing. The supply container is connected with the interior of the tattoo machine tip via a line in a portion in which said tattoo machine tip is held in the hand while tattooing. The line opens out into a colorant reservoir through which the needle is moved to wet the needle with sufficient ink for tattooing. In this tattoo machine tip too, the consumption of the liquid provided in the supply container is high.

From US 2002/0069726 A1, a tattoo machine tip has been known in which a storage tank (11) filled with ink is connected through a supply channel with a chamber (14) that can receive up to 2 ml of ink ([0031]) in order to be able to soak the tattoo needle (7) with ink.

From JP 2003/339875, it is known to use a liquid with which a tattoo can be removed in a device having a design comparable to that of a tattoo machine.

It is the object of the invention to provide a tattoo machine tip which is suitable for removing a tattoo and exhibits a low consumption of an agent required for removing the tattoo. Another object of the invention is to safely envelope the needle of a tattoo machine before and after use.

According to the invention, this object is achieved by the features of claim 1.

The tattoo machine tip according to the invention has a tip body with a needle channel for guiding at least one needle. The tattoo machine tip further has a supply container for receiving a liquid. According to the invention, the supply container is at least partly filled with a skin irritant. For wetting the needle with the skin irritant, the supply container is connected with the needle channel through a feed channel. For this purpose, the feed channel opens out into the needle channel so that there is a direct communication of the feed channel with the needle channel.

Since the supply container of the tattoo machine tip according to the invention contains skin irritants, the tattoo machine tip is suitable for removing tattoos or permanent make up. In the tattoo machine tip according to the invention, use is made of the fact that it is not required to introduce larger amounts of liquid to a depth of about 1 to 1.5 mm into the dermis of a human or animal skin, which is required in tattoo machine tips for tattooing. It is sufficient if relatively little skin irritant, as compared to the usual tattooing, is introduced into the epidermis, which has a thickness of about 0.5 mm, so that the color pigment agglomerates mechanically destroyed with the needle can enter the epidermis without being prevented therefrom by the natural healing process of the skin. From the epidermis, the color pigment agglomerates can be ablated along in the natural healing process of the skin. Since the skin irritant passes from the supply container through the feed channel directly into the needle channel, dead volumes as required in conventional tattoo machine tips for tattooing for sufficiently wetting the needle are avoided. In particular, liquid reservoirs provided within the tip body are not required. Instead, it is sufficient if one drop is delivered from the supply container into the needle channel to sufficiently wet the needle with the skin irritant. Thus, the liquid present in the supply container can be introduced into the skin almost completely without relevant residues remaining in the tattoo machine tip. The liquid consumption of skin irritant is minimized thereby. Further, the tip body of the tattoo machine tip can have a more stable design as compared with equally sized conventional tattoo machine tips since the wall thickness of the tip body can be higher due to the reduced void volume.

In “Lexikon der Hilfsstoffe für Pharmazie, Kosmetik und angrenzende Gebiete”, 4th Edition, 1996, Herbert P. Fiedler defines skin irritants under the keyword “skin irritations” to be chemical substances that exert a skin-irritating effect when contacted with human skin. Thus, skin irritants within the meaning of the present invention can have a primary irritant effect which occurs immediately upon contact with the skin. Skin irritants in which a damage is observed only after an extended time of action of the skin irritant substance in subthreshold doses in the form of a degenerative eczema are less suitable according to the present invention. Thus, it is particularly preferred according to the present invention to employ skin irritants that serve as skin-irritating agents.

Fiedler (supra) defines a scale for testing the skin-irritating effect that is officially recognized in the U.S. The most strongly skin-irritating substances are described as corrosive and highly dangerous, having to be provided with a warning notice. Such kinds of skin irritants will hardly be employed for the present invention, not in a pure form at any rate. The same applies to primary skin irritants, which are also highly dangerous and must bear warning notices. Skin irritants having the potential for a severe skin irritation are also less suitable according to the present invention. Skin irritants having no skin-irritating potential are not relevant in terms of wound healing, unless their presence as a filler of cells delays wound healing. Fillers include, for example, solids within the meaning of the present invention, such as common salt, diamond dust or quartz sand.

The skin irritants can be applied to the skin surface in a solid form and/or preferably in a liquid form or directly introduced into the fragments of color pigment agglomerates. According to the present invention, it is particularly preferred to employ skin irritant agents consisting of diluted aqueous solutions, dispersions and emulsions of the skin irritants. The chemical nature of the skin irritants is not restricted to a particular class of compounds. Thus, for example, diluted aqueous solutions of lactic acid, sodium hydrazide, common salt, amino acids or fruit acids, optionally containing small amounts of oxidants, may be employed. By simple series of experiments, their skin-irritating potential can be easily established, and thus an appropriate selection can be done.

Preferably, the supply container has a dosing means by which the skin irritant can be delivered in droplets and/or continuously or discontinuously. Thus, it is possible to deliver one drop each at a regular rate from the supply container into the needle channel, for example. Further, a continuous liquid flow can be adjusted. It is also possible to supply a defined amount of the skin irritant individually to the needle channel. The delivery of the skin irritant by means of the dosing means may be effected, for example, mechanically through a manually actuated piston. Alternatively, for example, compressed air, which exerts an adjustable constant pressure on a piston can be applied to avoid manual adjustments.

In particular, the dosing means has a conveying piston in order to press the skin irritant from the supply container into the needle channel. Thus, gravity flow, which changes as a function of the amount of skin irritant remaining in the supply container, is not required. Preferably, the conveying piston has an exterior contour that is matched in axial direction to both an interior contour of the supply container and an interior contour of the feed channel. Thus, the conveying piston can have a shape design to at least partially extend into the feed channel. Thus, the supply container and optionally also the feed channel can be discharged almost completely, so that non-utilized residual amounts of the skin irritant are almost completely avoided.

Preferably, the feed channel and the needle channel meet in such a way that the feed channel and the needle channel form an angle of from 10° to 90°, especially from 40° to 70°, preferably from 50° to 60°. In particular, the feed channel is inclined relative to the needle channel in such a way that the feed channel at least partially points towards the end of the tip body that faces the skin. Due to this arrangement of the feed channel relative to the needle channel, the skin irritant can be conveyed into the needle channel at least partially by gravity. Further, this enables the supply container to be at such a distance from the tip body that the tattoo machine tip can be handled easily without the supply container obstructing the view or the gripping of the tattoo machine tip being rendered difficult.

Preferably, the supply container is additionally connected with the tip body through stabilizing means, such as struts and/or ribs. In this way, the supply container can receive a correspondingly high amount of skin irritant without there being a risk that the supply container might break off the tip body, for example, in the area of the feed channel.

In order to facilitate the preparation of the tattoo machine tip, the supply container, the feed channel and the tip body and the stabilizing means, if any, can have an integral design. In this case, the supply container, the feed channel, the tip body and the stabilizing means are prepared by injection molding, in particular, and preferably consist of a plastic material.

Preferably, the tip body has a connecting means connected to said at least one needle by means of which the needle can be connected with a moving element of the tattoo machine. For example, said moving element is a plunger, a bar, a connecting rod or the like that provides an essentially translational reciprocating or up and down movement to prick and withdraw the needle or a set of several needles into and out of the skin. In particular, the connecting means is designed as an elastic body that has an insertion opening for frictional engagement with the moving element. Thus, the moving element of the tattoo machine can be simply engaged in the connecting means to provide a connection, especially rigid connection, with the needle. The moving element is preferably connected with said at least one needle rigidly, i.e., without a relative movement. In particular, the connecting means is guided within the tip body so that tilting of the needles or the moving element, for example, within the moving means, is avoided. More preferably, the connecting element has such a design that the moving element can be connected with the needle at a defined and/or constant distance from the needle. For example, the needle or needles and the moving element are rigidly connected with the connecting means, i.e., without a relative movement, for this purpose.

In particular, the tip body has an attachment means for attaching the tattoo machine tip to the tattoo machine. For example, the attachment means may be designed as a frictionally engaged plug connection, for example, by part of the tattoo machine tip being frictionally engaged in a reception opening of the tattoo machine. Preferably, the attachment means is designed as a bayonet mount to provide a particularly secure, detachable connection. Due to the attachment means, the tattoo machine tip can be offered as a separate accessory component of a tattoo machine. This enables the tattoo machine tip to be offered as a sterile or sterile-packed component, especially as a disposable article. This approach meets high hygienic demands, whereby the risk of infection with germ-contaminated needles, for example, is reduced.

The invention further relates to a device for removing a tattoo, comprising a housing in which a driving means is provided. A moving element guided within the housing is connected with said driving means. The device further comprises a tattoo machine tip that is further embodied as set forth above, in particular. The tattoo machine tip is connected with the housing and the moving element. The translational movement is forced by a suitable guide, in particular. However, the tip body of the tattoo machine tip and the housing of the device for removing a tattoo may also have an integral design and be prepared, for example, by injection molding a plastic material.

The driving means of the device preferably has an eccentric connected with a driving element of the driving means. By means of the eccentric, a rotational movement of the driving element, which may be the driving shaft of an electromotor, for example, can be converted to a translational movement of the moving element. Thus, in particular, the device for removing a tattoo comprises a tattoo machine tip connected with a tattoo machine and comprising an agent for removing a tattoo.

The device has such a design that the needle of the tattoo machine is arranged completely within the tip body when the driving means is in a switched-off state (idle state). This reduces the risk of getting hurt at the needle of the tattoo machine tip. This is achieved, for example, by using a restoring element to keep the needle and/or the moving element in a position in which the needle or needles do not project out of the tip body, but are located within the tip body. As the restoring element, for example, a spring is provided that exerts a force directed away from the end of the tattoo machine tip on said at least one needle and/or said moving element. The restoring element may also exert a force on the eccentric to keep it in a position in which the moving element is maximally remote from the end of the tattoo machine tip in the switched-off state of the driving means. Thus, the risk of infection, for example, of an HIV or hepatitis infection, is substantially prevented, especially when a needle is recapped.

In the following, the invention is further illustrated in a preferred embodiment thereof with reference to the attached drawings.

In the Figures:

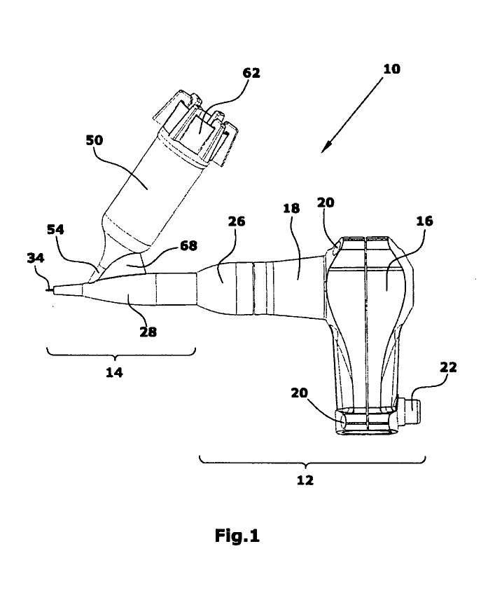

FIG. 1 shows a schematic and partially transparent side elevational view, drawn to scale, of a device for removing a tattoo;

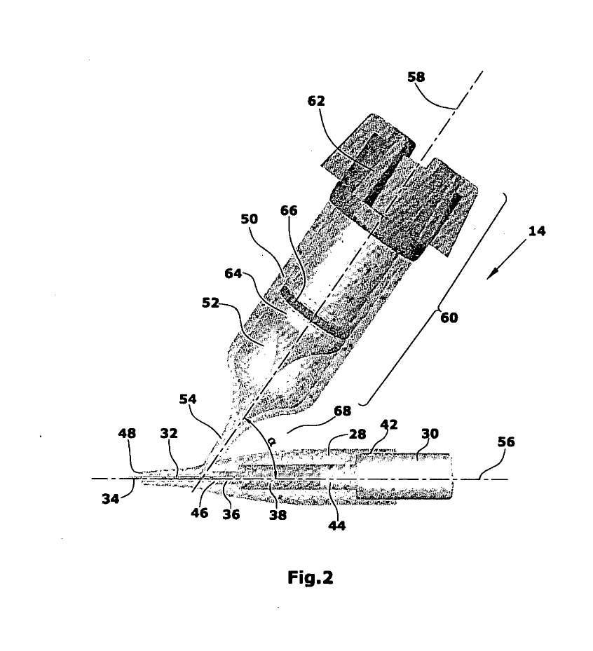

FIG. 2 shows a schematic, perspective and partially transparent side elevational view, drawn to scale, of a tattoo machine tip;

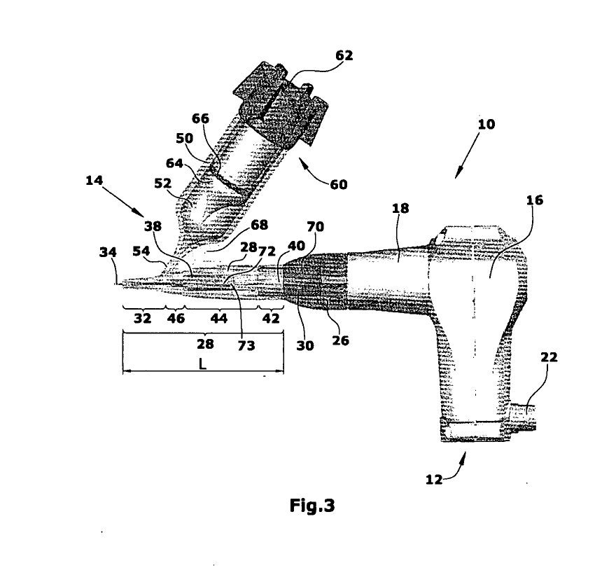

FIG. 3 shows a schematic, perspective and partially transparent side elevational view, drawn to scale, of a device for removing a tattoo with the tattoo machine tip in an assembled state; and

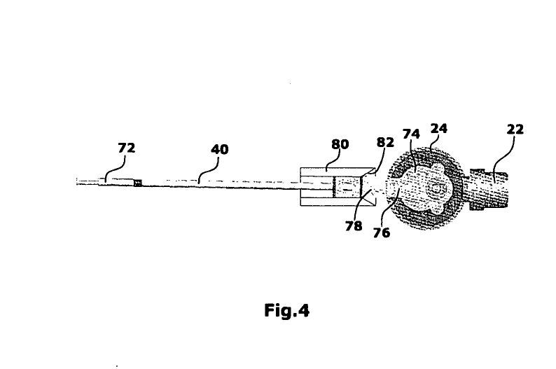

FIG. 4 shows a schematic top elevational view, drawn to scale, of a driving means of the device for removing a tattoo.

The device 10 according to the invention for removing a tattoo comprises a tattoo machine 12 and a tattoo machine tip 14 connected with said tattoo machine 12 (FIG. 1). The tattoo machine 12 has an upper shell 16 and a lower shell 18 that form a housing of the tattoo machine 12 and are interconnected by screws 20. In the upper shell 16, a port 22 is provided through which a driving means 24 of the tattoo machine 12 can be driven, for example, electrically or pneumatically. A bushing 26 through which the tattoo machine tip 14 is connected with the tattoo machine 12 is connected with the lower shell 18.

The tattoo machine tip 14 has a tip body 28 that has an attachment means 30 (FIG. 2). In the Example shown, the attachment means 30 is a plug connector which is firmly connected with the tip body 28 and can be frictionally engaged in the bushing 26 of the tattoo machine 12. The tip body 28 comprises a needle channel 32 in which several needles 34 are guided. The needles 34 may be conventionally available tattoo needles that may have a roughened surface. The needles 34 are interconnected at their ends facing away from the skin, for example, by soldering. This end is connected with a connecting element 36 which is in turn firmly connected with a connecting means 38, for example, by vulcanization, wherein said connecting means 38 may in turn be connected with a moving element 40 of the tattoo machine 12.

The tip body 28 has an attachment opening 42 in which the attachment means 30 is firmly inserted, for example, by a press fit. The attachment opening 42 is followed by a first guiding opening 44 in which the connecting means 38 is guided. The first guiding opening 44 is followed by a second guiding opening 46 in which the connecting means 36 is guided. This is followed by the needle channel 32, which has the smallest diameter as compared to the first guiding opening 44 and the second guiding opening 46. The needle channel 32 ends at a tip opening 48.

The tattoo machine tip 14 further has a supply container 50 filled with a skin irritant 52. On the exit side, the supply container 50 merges into a feed channel 54 that opens out into the needle channel 32. The disemboguement of the feed channel 54 into the needle channel 32 is effected under an angle α which is α=55° in the Example shown. That is to say, a center line 56 of the needle channel and a center line 58 of the feed channel form an angle of α=55°.

In the Example shown, the diameter of the needle channel 32 is chosen in such a way that five commercially available tattoo needles 34 are simultaneously guided in the needle channel 32. In the Example shown, the length of the needle channel 32 is chosen in such a way that the needles 34 can project from the tip opening 48 by about 1.5 to 2 mm. Preferably, the diameter of the feed channel 54 is chosen in such a way that the flow cross-section of the feed channel 54 corresponds to the effective flow cross-section of the needle channel 32, so that the flow rate in the feed channel 54 is essentially equal to the flow rate of the skin irritant 52 in the needle channel 32.

The supply container 50 further comprises a dosing means 60. The dosing means 60 comprises a cap 62 that can be screwed upon the supply container 50 and can be actuated quickly and easily with the left hand without the right hand having to let go off the tattoo machine tip 14. A piston 64 having an exterior contour matched to an interior contour of the supply container 50 and to an interior contour of the feed channel 54 is connected with cap 62. A gasket 66 of the piston 64 avoids leakage of the skin irritant 52 towards the cap 62.

For stably connecting the supply container 50 with the tip body 28, a stabilizing means 68 is provided which, in the Example shown, is a rib connected to the supply container 50, the feed channel 54 as well as the tip body 28. The supply container 50, the feed channel 54, the rib 68 and the tip body 28 form an integral component prepared by injection molding a plastic material.

In the assembled state (FIG. 3), the tattoo machine tip 14 is connected with the tattoo machine 12 through the attachment means 30. Additionally or alternatively, a bayonet mount may be provided for connecting the tattoo machine tip 14 with the tattoo machine 12. In the Example shown, the attachment means 30 comprises a passage opening 70 through which the moving element 40 is guided. At its tip, the moving element 40 has a connecting area 72 that is roughened, for example, by scoring. The connecting area 72 is engaged in an insertion opening 73 of the connecting means 38 and is thereby firmly connected with the connecting means 38. Preferably, the tip body has a length L which is chosen in such a way that the tip body 28 is gripped by the human hand when the device 10 is being used. Thus, the device 10 for removing a tattoo can be moved over the human skin like a paint brush.

The driving means 24 of the tattoo machine 12 comprises an eccentric 74 with whose driving element the moving element 40 is connected through a connecting rod 76 (FIG. 4). The connecting rod 76 is connected with the moving element 40 in an articulated manner through a second connecting means 78. The moving element 40 or the second connecting means 78 is guided in a guide 80, so that a purely translational movement is forced. The guide 80 has a bevel 82 facing towards the connecting rod 76 lest the connecting rod 76 should abut against the guide 80. The guide 80 may also be a component of the lower shell 18 of the body of the tattoo machine 12.

1. A tattoo machine tip for a tattoo machine (12), comprising:
a tip body (28) comprising a needle channel (32) for guiding at least one needle (34); and
a supply container (50) connected with said tip body (28) for receiving a liquid;
characterized in that
said supply container (50) is at least partially filled with a skin irritant (52), and said needle channel (32) is connected with said supply container (50) through a feed channel (54) opening out into the needle channel (32) for wetting the needle (34) with said skin irritant (52);
wherein said needle (34) is positioned completely within the tip body in the idle state.

2. The tattoo machine tip according to claim 1, characterized in that said supply container (50) has a dosing means (60) for delivering a drop and/or for continuously or discontinuously delivering said skin irritant (52).

3. The tattoo machine tip according to claim 2, characterized in that said dosing means (60) comprises a conveying piston (64) whose exterior contour is matched in axial direction to an interior contour of the supply container (50) and of the feed channel (54).

4. The tattoo machine tip according to claim 1, characterized in that said feed channel (54) and said needle channel (32) form an angle α of from 10° to 90°, especially from 40° to 70°, preferably from 50° to 60°.

5. The tattoo machine tip according to claim 1, characterized in that said supply container (50) and said tip body (28) are additionally interconnected through a stabilizing means (68), especially a strut and/or a rib.

6. The tattoo machine tip according to claim 1, characterized in that said supply container (50), said feed channel (54) and said tip body (28) and said stabilizing means (68), if any, have an integral design and, in particular, have been prepared by injection molding.

7. The tattoo machine tip according to claim 1, characterized in that said tip body (28) comprises a connecting means (38) connected with said at least one needle for connection with a moving element (40) with said tattoo machine (12), said connecting means (38), in particular, being designed as an elastic body that has an insertion opening (73) for frictional engagement with the moving element (40), and said elastic body is guided within said tip body (28).

8. The tattoo machine tip according to claim 7, characterized in that said connecting means (38) has such a design that the moving element (40) is connected with the needle (34) at a defined and/or constant distance from said needle (34).

9. The tattoo machine tip according to claim 1, characterized in that said tip body (28) comprises an attachment means (30) for attaching with a tattoo machine (12), especially one designed as a bayonet mount.

10. A device for removing a tattoo, comprising:
a housing (16, 18) in which a driving means (24) is provided;
a moving element (40) connected with said driving means (24) and guided within said housing (16, 18); and
a tattoo machine tip (14) according to claim 1 which is connected with said housing (16, 18) and with said moving element (40).

11. The device according to claim 10, characterized in that said driving means (24) comprises an eccentric (74) connected with a driving element for converting a rotational movement of the driving element to a translational movement of the moving element (40) by means of a guide (80).

12. The device according to claim 10, characterized in that the needle (34) and/or the moving element (40) are kept in a position in which the needle (34) is located completely within the tip body (28) by means of a restoring element in a switched-off state of said driving means (24).

13. The device according to claim 10, characterized in that said housing (16, 18) and said tip body (28) have an integral design.
